(19) United States
(12) Patent Application Publication
(10) Pub. No.: US 2016/0367500 A1
(43) Pub. Date: Dec. 22, 2016

(54) COMPOSITIONS COMPRISING VITAMIN C, VITAMIN E, AND COENZYME Q10 AND USE THEREOF FOR PROMOTING FEMALE FERTILITY AND REPRODUCTIVE HEALTH

(71) Applicant: FERTILIFY INC., Toronto (CA)

(72) Inventors: Cytlalli Dianne RUIZ-CHAPMAN, Toronto (CA); Antonio Franco MARRATO, Toronto (CA); Rolando RUIZ-CEPEDA, Mississauga (CA)

(21) Appl. No.: 15/187,318

(22) Filed: Jun. 20, 2016

Related U.S. Application Data
(63) Continuation-in-part of application No. PCT/CA2014/051187, filed on Dec. 9, 2014.
(60) Provisional application No. 61/918,759, filed on Dec. 20, 2013.

(51) Int. Cl. A61K 31/122; A61K 31/355; A61K 31/4745; A61K 9/00; A61K 31/714; A61K 31/59; A61K 31/519; A61K 31/375; A61K 31/05

(52) U.S. Cl. CPC A61K 31/122; A61K 31/375; A61K 31/355; A61K 31/4745; A61K 31/05; A61K 31/714; A61K 31/59; A61K 31/519; A61K 9/0053

(57) ABSTRACT

A new composition including substances that prolong and enhance the quality of the female reproductive system; effectively reducing the physiological effects of aging of the female reproductive system. In some aspects, the composition of the present invention includes coenzyme Q10, vitamin C, and vitamin E. As well, in some aspects, the composition of the present invention includes coenzyme Q10, resveratrol and pyrroloquinoline quinone (PQQ). In another aspect, the composition of the present invention further comprises vitamin D. In another aspect, the composition of the present invention comprises folic acid. In some aspects, the composition of the present invention increases the rate of fertility in women and promotes the development of the fetal nervous system.

### CROSS-REFERENCE TO RELATED APPLICATIONS

This is a continuation-in-part of PCT Application No. PCT/CA2014/051187 filed on Dec. 9, 2014 and published under WO 2015/089656, and claims priority from U.S. Provisional Patent Application No. 61/918,759 filed on Dec. 20, 2013, the entire contents of which are incorporated herein by reference.

### FIELD OF THE INVENTION

This invention relates in general to and more particularly to a composition, and a process of manufacturing a composition, consisting of a blend of supplements which offers reproductive and overall health benefits, and more particularly, for promoting reproductive health in females, namely fertility.

### BACKGROUND OF THE INVENTION

As financial, academic, professional and personal aspirations grow, many women delay the decision to begin conceiving. Commonly cited stresses leading to the decision to delay conceiving include the rising trends of extended education, the desire to establish a secure career, the need to obtain financial stability, the challenge of finding the right partner and the desire to explore life prior to conceiving children. Research in the United States has shown that women in 2012 gave birth at significantly older ages than women did in 1982, as summarized in FIG. 1 (Hamilton B. et al. (2013); Martin J. A. et al. (2009)). Further, more women are now giving birth between the ages of 30-34 than women between the ages of 20-24 (Hamilton B. et al. (2013)). Indeed, the gap in birth rates between these age groups has widened significantly in recent years, and this gap is expected to continue to grow.

However, a woman's decision to delay conception is confronted by research, which observes that a woman's ability to conceive is tied directly to her age. Specifically, it has been observed that as women age, the number and quality of eggs capable of creating a healthy child is reduced. Indeed, research has shown that by the age of thirty, women lose approximately 90% of their reserve of viable eggs (Wallace W. H. B. et al. (2010)). Additionally, in a recent In-Vitro Fertilization (IVF) study, it was demonstrated that the percentage of transferred embryos that resulted in a live birth drastically decreased as the egg donor ages past 35 (SART (2013)). This observation further supports the notion that the quality of a woman's eggs decreases with age and life style choices. Table 1 summarizes this study's findings.

TABLE 1
Percentage of embryo transfers resulting
in live births by age of woman

| | Percentage of embryo transfers |
|---|---|
| Age of woman | resulting in live births |
| Younger than 35 | 44.9% |
| Aged 35-37 | 37.3% |
| Aged 38-40 | 26.6% |
| Aged 41-42 | 15.2% |
| Aged 43-44 | 6.7% |

Adapted from: SART (2013).

Therefore, fertility in women is closely related to reproductive age and becomes significantly compromised before the onset of premenopausal menstrual irregularity. It has been observed that female fertility peaks at the age of 25, and after the age of 35 suffers a rapid decline (SART (2013)). The reduced likelihood of fertility as women age has given rise to many potential solutions to the problem, ranging from medical-based approaches, such as Assisted Reproductive Technologies (ART), to unproven-alternative approaches, both of which attempt to improve a woman's ability to conceive.

Medical-based approaches, such as ART, have been backed by scientific research, whereas unproven-alternatives remain speculative. Such medical-based solutions include, but are not limited to, medications, IVF, surrogacy and many other specialized techniques. These options, however, can prove to be financially expensive as well as emotionally and physically trying.

Unproven-alternatives to medical-based interventions include, but are not limited to, holistic approaches and unconventional medicine. While these options can be less costly and may have an appeal when compared to medical-based approaches, they typically lack significant objective scientific support of their efficacy.

The limitations of both medical-based and unproven-alternative fertility treatment approaches have given rise to the need for a new set of solutions that are scientifically backed, non-medical, natural, affordable, and that would address the following scenarios:
1. Women who are not yet ready to conceive but wish to be proactive in preserving their reproductive health for the future by natural means;
2. Women, with no fertility issues, who are currently ready to conceive and wish to increase the likelihood of successful conception;
3. Women, with fertility issues, who are currently ready to conceive and wish to increase the likelihood of successful conception;
4. Women, with fertility concerns, who choose to pursue some form of assisted treatment, either medical-based or unproven-alternatives, and wish to increase the likelihood of success of the treatment.

Therefore, there is an unmet need for an effective method of prolonging or supporting female fertility in women as they age, promoting the development of a healthy fetus in prospective mothers to achieve a successful conception, and enhancing existing fertility in women as they attempt to conceive.

Thus, a treatment that can be administered to women at any age, typically after reaching sexual maturity, that reduces ageing of the female reproductive system, and that provides essential nutrients required for optimal reproductive function, would be of considerable benefit. The present invention discloses such a composition with these possible advantages.

### SUMMARY OF THE INVENTION

In accordance with one aspect of the present invention, the following combination of supplements provides an effective treatment to enhance and prolong the effectiveness of the female reproductive system, as well as supply essential nutrients required for optimal reproductive function.

In one aspect of this invention, there is provided a composition comprising: (a) coenzyme Q10 in the range of 50-1200 mg, (b) vitamin C in the range of 50-2000 mg, and (c) vitamin E in the range of 10-1000 mg.

In another aspect of this invention, there is provided a composition comprising: (a) coenzyme Q10 in the range of 50-1200 mg, (b) PPQ (pyrroloquinoline quinone) in the range of 10 to 300 mg, and (c) Resveratrol in the range of 20 to 300 mg.

In another aspect of this invention, there is provided a composition for female fertility, the composition comprising coenzyme Q10, vitamin C, and vitamin E. In another aspect, the composition comprises coenzyme Q10, PPQ, and Resveratrol.

In yet another aspect there is provided the composition of the present invention for female fertility, further comprising Vitamin D in the range of 200-4000 IU.

In yet another aspect there is provided the composition of the present invention for female fertility, further comprising folic acid in the range of 0.1-5 mg.

In yet another aspect there is provided the composition of the present invention for female fertility, further comprising vitamin B12 in the range of 2.0 mcg-1000.0 mcg.

In yet another aspect there is provided for the composition of the present invention, an oral daily dose in unit form.

A potential example of the embodiment of the present invention is detailed in Table 2.

TABLE 2
Illustration of potential embodiments of present invention

| | Amounts per ingredient |
|---|---|
| Ingredient | in potential embodiment |
| Coenzyme Q10 (mg) | 300 |
| Vitamin C (mg) | 250 |
| Vitamin E (mg) | 250 |
| Vitamin D (IU) | 1,000 |
| Folic Acid (mg) | 0.5 |
| Coenzyme Q10 (mg) | 300 |
| Vitamin C (mg) | 85 |
| Vitamin E (mg) | 15 |
| Vitamin D (IU) | 1,000 |
| Folic Acid (mg) | 1 |
| PQQ (mg) | 20 |
| Resveratrol (mg) | 40 |

Possible advantages of the present invention include the reduction of ageing of the female reproductive system. This is mediated through a reduction of the contribution of Reactive Oxygen Species (ROS) to cellular damage (known as the physiological effect of aging) in the female reproductive system using enzymatic and non-enzymatic antioxidants, the regulation of hormones, including but not limited to Anti-Müllerian hormone (AMR), involved in promoting female fertility using specific supplements, and the promotion of the proper development of the fetal nervous system through the use of specific supplements.

### BRIEF DESCRIPTION OF THE DRAWINGS

A detailed description of the preferred embodiments is provided herein below by way of example only and with reference to the following drawings, in which:

Table 1, which illustrates the percentage of embryo transfers resulting in live births.

Table 2, which illustrates the potential levels of composition components in accordance with a preferred embodiment of the present invention.

Table 3, which illustrates the tolerable upper limit doses of coenzyme Q10, vitamins C, E and D, and folic acid.

FIG. 1, which illustrates the percentage change in births per 1,000 women in the United States of America, by women's age groups, 1982 versus 2012. (Adapted from: Hamilton B. et al. (2013) and Martin J. A. et al. (2009))

FIG. 2, which illustrates the role of coenzyme Q10 in the transport of electrons and protons and the regulation of ROS, and in in cellular energy and ROS production. (Adapted from: Bentov Y. et al. (2013) Aging oocyte and mitochondrial function. Fertil Steril.) The electron transport chain that supports the process of oxidative phosphorylation (OXPHOS) in the mitochondria. This illustration shows the five complexes and the flow of electrons and protons as well as the central role of oxidized coenzyme Q10 (CoQ10) as an electron and proton transporter; describes the sites of reactive oxygen species (ROS) production and the Q cycle; and identifies the central role of CoQ10 in the flow of electrons and protons, showing how the associated increase ROS in a deficiency in CoQ10 leads to a compensatory increase in the number of sites producing ROS. Also shown is the proximity of TOS production sites to the energy-producing proteins and mitochondrial DNA.

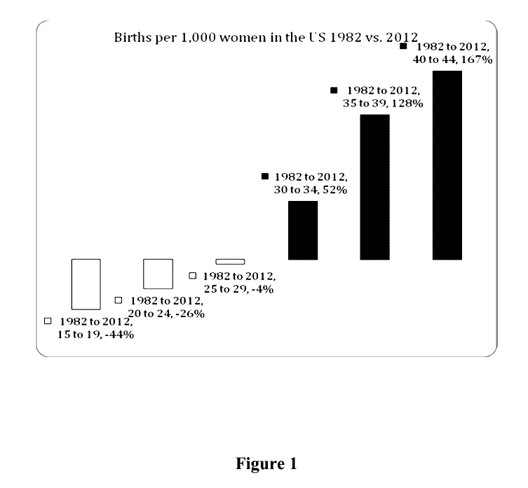

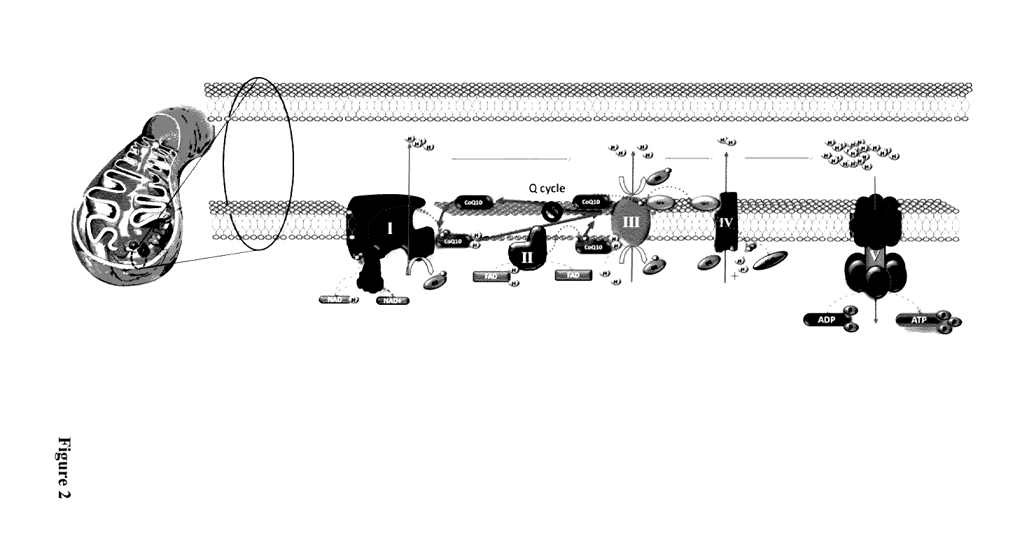

In the drawings, preferred embodiments of the invention are illustrated by way of example. It is to be expressly understood that the description and drawings are only for the purpose of illustration and as an aid to understanding, and are not intended as a definition of the limits of the invention.

### DETAILED DESCRIPTION OF THE PREFERRED EMBODIMENT

In one aspect of this invention, there is provided a composition comprising: (a) coenzyme Q10 in the range of 50-1200 mg, (b) vitamin C in the range of 50-2000 mg, and (c) vitamin E in the range of 10-1000 mg.

In another aspect of this invention, there is provided a composition comprising: (a) coenzyme Q10 in the range of 50-1200 mg, (b) PPQ (pyrroloquinoline quinone) in the range of 10 to 300 mg, and (c) Resveratrol in the range of 20 to 300 mg.

In another aspect of this invention, there is provided a composition for female fertility, the composition comprising coenzyme Q10, and vitamin C, vitamin E, PPQ, Resveratrol, or combinations thereof.

In another aspect, there is provided the composition of the present invention wherein coenzyme Q10 is in the range of 50-1200 mg.

In yet another aspect, there is provided the composition of the present invention wherein Vitamin C is in the range of 50-2000 mg.

In yet another aspect, there is provided the composition of the present invention wherein Vitamin E is in the range of 10-1000 mg.

In yet another aspect, there is provided the composition of the present invention wherein PPQ is in the range of 10 to 300 mg.

In yet another aspect, there is provided the composition of the present invention wherein Resveratrol is in the range of 20 to 300 mg.

In yet another aspect, there is provided the composition of the present invention wherein the composition comprises coenzyme Q10 in the range of 50-1200 mg, vitamin C in the range of 50-2000 mg, and vitamin E in the range of 10-1000 mg.

With reference to Table 3, it is established that coenzyme Q10 is safely tolerable up to 1200 mg per day (Mayo Clinic (2013)). Furthermore the tolerable upper limit of intake of vitamin C and vitamin E is 2000 mg and 1000 mg per day, respectively (Health Canada Nutrition Tables (2013)). Furthermore, studies have shown that resveratrol is safe to take consecutively at 1,000 mg doses per day (Brown V., et al. (2010)). Furthermore, several studies have shown that PQQ is safe to take in the 10 mg to 20 mg per day dose. The NHPD has also approved the use of vitamin E and vitamin C for the maintenance of good health (NHPD monograph: vitamin E and C).

TABLE 3
Tolerable upper limits of ingredients

| | Health Canada suggested daily intake levels for | | | |
|---|---|---|---|---|
| | females aged 19-50 | | | Suggested daily intake |
| | Estimated | Recommended | Tolerable | levels for adults |
| | average | dietary | upper | over the age of 18 |
| Ingredient | requirement | allowance | intake level | Suggested dosage level |
| Coenzyme Q10 (mg) | | | | 50-1,200 |
| PQQ (mg) | | | | 10-20 |
| | | | | (no known upper limit) |
| Resveratrol (mg) | | | | 20-1,000 |
| Vitamin C (mg) | 60 | 75 | 2,000 | |
| Vitamin E (mg) | 12 | 15 | 1,000 | |
| Vitamin D (IU) | 400 | 600 | 4,000 | |
| Folic Acid (mg) | 0.16 | 0.2 | 0.5 | |

Adapted from: Health Canada Nutrition Tables (2013), Mayo Clinic (2013) and FDA (2015).

Female fertility has been observed to decline significantly at thirty years of age in human females, with an even steeper decline occurring after the age of thirty-five (Broekmans F. J. et al. (2007)). This culminates in menopause at the ages of between 50 and 51. This research demonstrates the effects of age on female fertility. Significantly, the modern tendency for women to postpone childbearing to the their thirties, and later, has made reproductive ageing, as the phenomenon is known, a significant issue for governmental health care systems (Balasch J. et al. (2011)).

It is well known that the intra-cellular processes for energy generation, specifically aerobic metabolism, leads to the production of toxic metabolic by-products, in particular Reactive Oxygen Species (ROS), which interferes with key regulatory mechanisms and often damages biomolecules both intracellularly and extracellularly, leading to ageing of tissues and the broader organism as a whole (Harman D. (2006)). ROS include superoxide anion radicals, hydroxyl radicals, and hydrogen peroxide. The source of ROS-mediated damage has been demonstrated to be leakage of electrons from the inner mitochondrial membrane of a cell during oxidative phosphorylation and generation of adenosine triphosphate (ATP). More specifically, in steroidogenic tissues such as the ovary, steroidogenic cytochrome P450 enzymes are also a relevant source of ROS, and together these sources lead to ageing of the female reproductive system. (Finkel T. et. al. (2000)).

The female reproductive system is defined as comprising, but not limited to; fallopian tubes, ovaries, uterus, cervix, vagina, oocytes, granulosa cells, and ovum.

As described above, ROS is waste created by cells as a by-product of cellular energy generation. As ROS increases, damage to the cell increases, cell quality decreases and physiological ageing occurs (Liochev (2012)). Intracellular and extracellular ROS levels can also increase as a consequence of exposure to toxins associated with smoking, poor diet, radiation, and other sources (Trifunovic et al. (2004)).

A leading theory regarding the age-related decline in female fertility and its effects on both somatic cells and oocytes suggests that point mutations and deletions in mitochondrial DNA (mtDNA) accumulate over time in a cell's mitochondria as a consequence of ROS-mediated cell damage. These mutations are unevenly distributed, can accumulate clonally, and can cause a mosaic pattern or respiratory chain deficiencies in tissues characterized by energy consumption. This is particularly profound in cells with a larger number of mitochondria. Furthermore, oocytes have the largest number of mitochondria and mtDNA copies of any cell, at least 1 to 2 orders of magnitude more than somatic muscle and neuron cells that have high-energy requirements (Trifunovic et al. (2004)).

Supporting the notion that point mutations and deletions in mtDNA significantly compromise female fertility, a murine model study by Trifunovic et al. observed that mice with a threefold to fivefold increase in the level of point mutations and deletions of mtDNA demonstrated a profound reduction in fertility (Trifunovic et al. (2004)).

Critically, it has been observed that an increase in ROS-mediated ageing has also been correlated with the age-dependent decrease in tissue concentrations of the molecule coenzyme Q10. Coenzyme Q10 is a lipophilic enzymatic molecule synthesised within the mitochondria and is an essential element in anti-oxidant defence. It has been experimentally observed that there is a gradual, age-related decline in coenzyme Q10 tissue concentrations that is worsened by the use of certain commonly used drugs, including the class of drugs known as statins. Coenzyme Q10 deficiencies, often caused by genetic mutations of the genes encoding coenzyme Q10's synthesis in the mitochondria, have also been shown to lead to the dysfunction of mitochondria in the nervous system, skeletal muscles and endocrine glands (Quinzii C. M. (2007)).

ROS-mediated female reproductive system ageing is therefore related to decreasing coenzyme Q10 tissue concentrations. Studies have shown a significant decline in the expression of the enzymes involved in coenzyme Q10 production in aged cumulus cells. The decline in expression of enzymes related to coenzyme Q10 production correlates with lower levels of coenzyme Q10, which in turn is tied to lower fertility rates (Quinzii C. M. (2007)).

More importantly, it has also been shown that treatment with CoQ10 significantly increases the number of ovulated eggs after stimulation in aged female mice. In experimental models of 52-week-old mice, coenzyme Q10-treated female mice had significantly larger litter sizes than control groups who did not receive coenzyme Q10 treatment (Bentov Y. et al. (2013)).

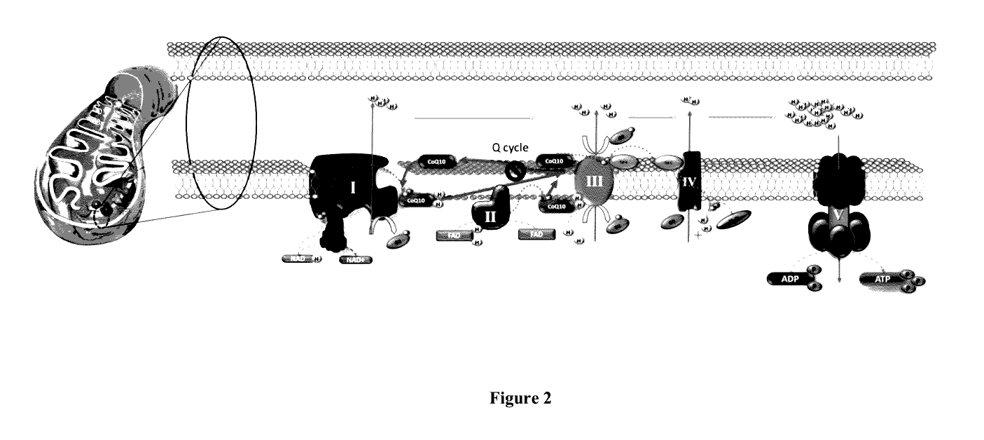

A comprehensive study conducted by Bentov et al. summarized the critical role ROS plays in cellular ageing, and the coenzyme Q10-mediated mechanism that removes ROS from the mitochondria, depicted in FIG. 2. Specifically, it was proposed that coenzyme Q10-mediated removal of mitochondrial ROS helps prevent mitochondrial mutations and cellular damage, increasing the quality of the cell (Bentov Y. et al. (2013)). Further, the removal of ROS from the mitochondria has significance for the accumulation of mutations in the context of cellular replication. The removal of ROS aids in preventing mutations from accumulating in new generations of cells as the cells and their genetic mutations proliferate, further reducing the effects of ROS-mediated ageing of the female reproductive system (Bentov Y. et al. (2013)).

The composition of the present invention also comprises the non-enzymatic molecules vitamin C and vitamin E, which have been experimentally demonstrated to play a significant role in reducing ROS-mediated damage, and therefore ageing, of the female reproductive system. Vitamin C is a known redox catalyst that can reduce and neutralize ROS (Agarwal A. et al. (2012)). Vitamin E is a lipid soluble vitamin with antioxidant activity. It plays a major role in antioxidant activities because it reacts with lipid radicals produced during lipid peroxidation (Agarwal A. et al. (2012)). Together, these molecules possess anti-oxidant activity. For example, in studies conducted using ex-vivo murine models, vitamin C and vitamin E improved IVF and increased the rate of embryo development, directly reversing the effect of ROS-mediated embryo-toxicity in cultures (Xia W. (2002)).

The composition of the present invention also comprises PQQ (pyrroloquinoline quinone) and/or Resveratrol. PPQ protects the mitochondria from oxidative stress (ROS (Reactive Oxygen Species) damage) and works in conjunction with CoQ10 to protect against ROS damage. Researchers at the University of California at Davis released a study showing that PQQ's critical role in growth and development stems from its unique ability to activate cell signaling pathways directly involved in cellular energy metabolism, development, and function (Chowanadisai, W. et al. (2010)). The study demonstrated that PQQ not only protects mouse hepatocyte mitochondria from oxidative stress, it promotes the spontaneous generation of new mitochondria within aging cells, a process known as mitochondrial biogenesis (Chowanadisai, W. et al. (2010)). Considering that oocytes have orders of magnitude more mitochondria than any other energy intensive cell, the spontaneous promotion of new mitochondria in the aging oocyte would be greatly beneficial to fertility. The University of California at Davis study also shows us that PQQ also helps to protect against Reactive Oxygen Species (ROS) damage, aiding in the efforts of coenzyme Q10 (Chowanadisai, W. et al. (2010)).

Resveratrol protects against mitochondrial dysfunction, and helps defend against oxidative stress. Resveratrol is an effective reactive oxygen species scavenger that exhibits an antioxidative property (Liu, F. et al (2015)). Resveratrol works with CoQ10, to help promote healthy mitochondrial function.

Methylglyoxal, a reactive dicarbonyl compound, is mainly formed from glycolysis. Methylglyoxal can lead to the dysfunction of mitochondria, the depletion of cellular anti-oxidation enzymes and the formation of advanced glycation ends. Previous studies showed that the accumulation of methylglyoxal and advanced glycation ends can impair the oocyte maturation and reduce the oocyte quality in aged and diabetic females. A study by Lui Y., et al., shows that resveratrol, a kind of phytoalexin found in the skin of grapes, red wine and other botanical extracts, can alleviate the adverse effects caused by methylglyoxal, such as inhibition of oocyte maturation and disruption of spindle assembly. Besides, methylglyoxal-treated oocytes displayed more DNA double strands breaks and this can also be decreased by treatment of resveratrol. Further investigation of these processes revealed that methylglyoxal may affect the oocyte quality by resulting in excessive reactive oxygen species production, aberrant mitochondrial distribution and high level lipid peroxidation, and resveratrol can block these cytotoxic changes. (Liu Y., et al. (2013))

Once CoQ10 removes the ROS from the mitochondria, vitamin C and E help to scavenge and neutralize ROS from cellular environment, preventing ROS-mediated cellular damage.

More specifically, free radicals, atoms with an unpaired electron such as ROS, are neutralized of their actions by antioxidants such as vitamins C and E (Devasagayam et al. (2004)). Vitamin E has consistently been recognized as possessing significant antioxidant activity, having been shown to function as a chain-breaking antioxidant which prevents the propagation of free radical reactions (Devasagayam et al. (2004)). Similarly, vitamin C has been recognized as having a central anti-oxidant role in the cell (Devasagayam et al. (2004)). Studies have shown that vitamin C is a primary anti-oxidant responsible for neutralizing ROS-mediated damage and protecting cells against death from oxidative stress (Guaiquil et al. (2001)). Both vitamin C and E therefore possess significant protective activity against the harmful effects of ROS (Devasagayam et al. (2004)).

Therefore, a possible advantage of the present invention is the reduction of ageing of the female reproductive system, thus prolonging fertility in women as they age.

In yet another aspect, the composition of the present invention, the composition further comprising Vitamin D in the range of 200-4000 IU.

In yet another aspect, the composition of the present invention wherein Vitamin D enhances fertility rates.

Vitamin D promotes internal health. The NHPD has approved the use of vitamin D for the maintenance of good health (NHPD monograph: vitamin D). More specifically, vitamin D has been demonstrated to aid the regulation of the production and secretion of hormones involved in fertility at the glandular level. Furthermore, the tolerable upper limit of vitamin D intake in humans is 4000 IU, per day (Health Canada Nutrition Tables, (2013)).

Vitamin D receptors are found in various reproductive tissues, including ovarian and uterine tissue (Yoshizawa T. et al. (1997); Kinuta K. et al. (2000)). Studies have shown normal growth and development before weaning (analogous to human puberty) in mice lacking the vitamin D receptor; however, after weaning, these mice failed to thrive and were infertile. Vitamin D has also been shown to regulate expression of the Hox gene in the uterus (Daftary G. S. et al. (2006)). Vitamin D and the transcription factors produced from the Hox genes, specifically HOXA10/11, function as part of the endocrine signal-transduction pathway, regulating endometrial development in preparation for implantation (Daftary G. S. et al. (2006)).

In-vivo murine models support the above-mentioned in-vitro research. In an early study by Halloran and Deluca, female rats were fed diets that were sufficient or deficient in vitamin D. The rats were then mated and their fertility determined. The authors reported that rats fed the diet deficient in vitamin D had 75% reduced fertility and 30% smaller litter sizes than rats fed the vitamin D sufficient diet (Halloran B. P. et al. (1980). Hickey and colleagues also found that female rats fed a diet lacking vitamin D before mating had significantly smaller litters than female rats fed a diet containing vitamin D (Hickie J. P. et al. (1983)).

These findings extend to human clinical studies as well. Fertility researchers at Mount Sinai Hospital studied 173 Canadian women between the ages of 18 and 41 who were all undergoing IVF treatment. After accounting for patient IVF cycles, the demographics amongst participants and the varying vitamin D levels amongst the female participants in the study, the researchers observed that 52.5% of women with sufficient levels of vitamin D had significantly higher pregnancy rates per IVF cycle, compared with the 34.7% whose levels were insufficient or deficient (Gardedian K. et al. (2013)). Therefore, sufficient levels of Vitamin D resulted in improved fertility rates in women when compared to deficient levels.

In more recent studies, women with sufficient levels of vitamin D have also shown increased levels of anti-Müllerian hormone (AMH) (Dennies et al. (2012)). AMH is a hormone that is measured as a proxy to determine true ovarian reserve. Indeed, AMH levels have been credited as the most reputable and effective means to assess ovarian reserve (Toner et al. (2013)). In assessing ovarian reserve, higher levels of AMH indicate high ovarian reserve; the opposite is true for low levels of AMH.

AMH is secreted by the granulosa cells of ovarian follicles and is responsible for the formation of primary follicles (Weenen C. et al. (2004)). AMH is present in females from the onset of puberty until menopause, and regulates follicular recruitment and development within the ovary (Dennis et al. (2012)). Low AMH levels have been correlated with poor success rates of assisted reproductive technology (Dennis et al. (2012)). Further, AMH levels have been demonstrated as possessing the strongest correlation with the number of retrieved oocytes (Dennis et al. (2012)). Vitamin D is a key promoter for the AMH response, and is therefore thought to be a regulator for AMH concentration (Dennis et al. (2012).

A possible advantage of the present invention is the vitamin D-mediated enhancement of fertility rates in women, in part through the regulation of fertility-associated hormones, including but not limited to AMH. Enhancement may be defined as increasing or improving the existing fertility rate of women.

In yet another aspect, the composition of the present invention further comprises folic acid in the range of 0.1-5 mg. The composition of the present invention may further include vitamin B12 for the improved uptake of folic acid. Typically, the ranges of vitamin B12 is between 2.0 mcg-1000.0 mcg, namely the daily recommended intake of 2.4 mcg.

In yet another aspect, the composition of the present invention wherein folic acid promotes the development of the nervous system of a fetus.

The NHPD has also approved the use of folic acid for the maintenance of good health (NHPD monograph: folic acid). Further, folic acid has been strongly implicated in the development of the fetal central nervous system. A study published in the New England Journal of Medicine conducted an analysis on a study population that included live births, stillbirths, and the termination of pregnancies because of fetal anomalies among women residing in seven Canadian provinces from 1993 to 2002. The study considered the role folic acid had in the development of the fetal central nervous system by evaluating the diets of women based on the degree of folic acid fortification occurring in the diet prior to and during pregnancy. Furthermore, fortification of food with folic acid was associated with a significant reduction in the rate of neural-tube defects in the study population (De Walls P. et al. (2007)).

The inclusion of folic acid is a proactive approach for both women trying to conceive as well as women that are not yet ready to conceive. Approximately 50% of all pregnancies are unintended in the United States of America, and women who may not yet be ready to conceive have a 50% chance of unexpectedly becoming pregnant (Finer L. et al. (2011)). Keeping in mind folic acid's role in the proper development of a fetus, it is therefore important that women, who are not yet ready to conceive, be proactive in case pregnancy unexpectedly occurs.

A possible advantage to the present invention, as a consequence of the inclusion of folic acid in the composition, is the promotion of the development of a fetal central nervous system in fertile women, whether or not the woman is attempting to conceive at the time of taking the composition. The inclusion of vitamin B12 with folic acid may further impact the development of the spine and central nervous system of a fetus.

In yet another aspect of the composition of the present invention, an oral daily dose in unit form.

In yet another aspect of the composition of the present invention, the oral daily unit dosage form selected from the group consisting of: a capsule, a tablet, a chewable tablet, a liquid, a liquid-gel capsule, a gel capsule with dry powder, a vegetable capsule with dry powder, a dry powder, an effervescent tablet or a gum-based chewable.

In yet another embodiment, a pack comprising an oral daily dose in unit form wherein the oral daily unit dosage form is selected from the group consisting of: individual capsules, tablets, chewable tablets, liquid-gel capsules, gel capsules, vegetable capsules, effervescent tablets and or gum-based chewables.

The composition of the present invention is manufactured using a number of processes, each of which start with the sourcing of either all-natural, organic or synthetic supplement ingredients which include the various forms of CoQ10 (ubiquinone, ubiquinol), the various vitamers of vitamin E (tocopherols (d-alpha, d-beta, d-gamma, and d-delta-tocopherol), tocotrienols), the various vitamers of vitamin D (ergocalciferol (D2), vitamin D3 (cholecalciferol)), the various vitamers of vitamin C (ascorbic acid, calcium ascorbate, sodium ascorbate, other salts of ascorbic acid) and the various forms of folic acid (vitamin B-9, folinic acid (leucovorin), 5-Methyltetrahydrofolate).

The supplemental ingredients may also include PQQ and it could be used with, or as a substitute for Vitamins C and/or E. The supplemental ingredients may further include Resveratrol, and could be used with or in place of Vitamins C and/or E.

The dry forms of these ingredients are then mixed corresponding to the specific volumes required in the composition of the present invention's formula, also with respect to the number of units to take per dosage, to create a mixture. The mixture is then either pressed into tablet form (to create a tablet), encapsulated into a gel-capsule or vegetable based capsule, mixed with all natural, organic or synthetic flavouring (to create a chewable) or compressed into an airtight container (effervescent).

The wet forms of these ingredients undergo the same mixture volumes as their dry form counter-parts; however, the wet forms can be left in liquid form and packaged in bottles, injected into liquid gel-capsules or cured in moulds to create gum-based chewables.

The tablet, gel-capsule, vegetable based capsule, chewable or effervescent forms of these ingredients can also be combined to the specific volumes required in the composition of the present invention's formula, also with respect to the number of units to take per dosage, to create a pre-packed dosage. The individual ingredients are then collected and pre-packaged together in either a pillow pack, bottle or other container, as a single dose.

The various forms of the composition of the present invention are then bottled (if not already bottled as in the pure liquid form), and sealed.

More generally, compositions according to the present invention may be readily prepared according to standard procedures known in the art for the preparation of compositions for oral administration. Capsules may contain colorants such as a chlorophyliin copper complex, which serves as a colouring agent for the capsule shell. Extracts may be prepared according to standard procedures known in the art for the preparation of plant extracts. Vitamins may also be obtained from commercial sources ready to be blended into the compositions of the present invention from a variety of commercial suppliers.

The present invention will be understood by reference to the following non-limiting examples:

In creating the following examples of the invention, it is necessary to source the various ingredients from vendors that offer the desired quality and form of the ingredient. Ingredient qualities can include, but are not limited to, synthetic, all natural, certified organic, and other similar qualities. Ingredient forms can include, but are not limited to, dry powder, tablet, liquid, and other similar ingredient forms.

### Example 1

In this instance of the invention, the form of the invention will be a tablet (chewable or non-chewable) or dry powder. To create the tablet, the ingredients, if not already in powder form, are crushed into powder. The separate ingredient powders are then dried in an apparatus to remove any undesired moisture from the powders. If required, additional ingredients such as binding agents, flavourings, colourings, effervescence agents, and other additional ingredients may also be included. The powders are then measured to the required dosage per ingredient, per unit. If left as a powder, the units would then be packaged. If the final product were to be a tablet, then the units would be put into a compression apparatus. The compression apparatus will then gather the various measured powders and press them into a form. The form is sized with respect to the per unit volume of each ingredient to offer the proper dosage, as well as to be palatable for human consumption. In some instances the dosage per tablet can be a fraction of the invention's recommended daily dosage, and the consumer of the tablet would be instructed to take a multiple of tablets, which equals the total recommended daily dosage. This would likely be done in the case where the daily recommended dosage is too great to be palatable in one unit. Once pressed and formed, if desired, the units can then be coated by being placed into a coating apparatus, which evenly distributes a coating over the surface of each unit. Surface coating maybe used for colour, increased hardness, easy-swallow coating, etc. The units are then aligned and quality checked for weight and shape as well as for any abnormalities. Units that do not pass the quality check are discarded. Units that pass the quality check are counted and collected into bottles, or other suitable package forms, via a packaging or bottling apparatus.

### Example 2

The invention may also be in the form of a gel based or vegetable based two-piece capsule. In manufacturing such a capsule, the same process as utilized in Example 1 is used, however, instead of pressing the ingredients into a form, the capsules are opened, the ingredients are filled into one half of the capsule, and the capsules are reconnected. A filling apparatus completes this aspect of the manufacturing process. The capsules are sized with respect to the per unit volume of each ingredient to offer the proper dosage, as well as to be palatable for human consumption. In some instances the dosage per capsule can be some fraction of the invention's recommended daily dosage, and the consumer of the capsule would be instructed to take a multiple of capsules, which equals the total recommended daily dosage. This would likely be done in the case where the daily recommended dosage is too great to be palatable in one unit. The filled capsules are then checked for quality by such measures as weight, or any abnormalities. The capsules are then counted and packaged into bottles or other suitable package forms by a bottling or packaging apparatus.

### Example 3

In the instance where the invention is in the form of a liquid, the liquid versions of each ingredient will be quality checked, then measured for required dosage per unit. The liquids will then be combined in whole, or in per unit part according to the required dosage composition. Other ingredients may be included in the composition such as mixing agents, colourings, flavourings, etc. The liquid would then be checked for quality. The liquid would then be filled into bottles, or other suitable package forms, by a bottling or packaging apparatus.

### Example 4

In the instance where the invention is in the form of a liquid gel, single piece capsule, the liquid form of the composition as detailed in Example 3 would be injected into a gelation-based capsule by a gel encapsulation apparatus. If required, other liquids may be included to the mixture, such as mixing agents, etc. The capsules are sized with respect to the per unit volume of each ingredient to offer the proper dosage, as well as to be palatable for human consumption. In some instances the dosage per capsule can be some fraction of the invention's recommended daily dosage, and the consumer of the capsule would be instructed to take a multiple of capsules, which equals the total recommended daily dosage. This would likely be done where the daily recommended dosage is too great to be palatable in one unit. The gel capsules may then require further treatment to ensure proper capsule strength and form. The capsules are then checked for quality. The units are then counted and packaged into bottles or other suitable package forms by a bottling or packaging apparatus.

### Example 5

In the instance where the invention is a gum-based chewable, the liquid and or dry powder versions of each ingredient will be quality checked, then measured for required dosage per unit. The liquids and or powders will then be combined in whole, or in per unit part according to the required dosage composition. Other ingredients may be included in the composition such as mixing agents, colourings, flavourings, gum-based binding agents, etc. The mixture would then be placed into moulds to form the individual units. The mixture would then be cured in a curing apparatus to ensure solid form. The units are then checked for quality. The units are then counted and packaged into bottles or other suitable package forms by a bottling or packaging apparatus.

### Example 6

In the instance where the invention is a combination of single and or mixed ingredient units placed into a pillow package, or other suitable package, the ingredients must first be sourced in dosages that will sum to the required dosage pre unit. Then each of the ingredient tablets, capsules, gel capsules, etc. will be combined on a per dosage bases into single serving pillow packages, or other suitable packages.

In the above-mentioned examples, upon completion of bottling or any other suitable packaging process, the product will then be labelled and optionally packaged with suitable external packaging.
It will be appreciated by those skilled in the art that other variations of the preferred aspect may also be practised without departing from the scope of the invention.

Other variations and modifications of the invention are possible. All such modifications or variations are believed to be within the sphere and scope of the invention as defined by the claims appended hereto.

I claim:

1. A composition comprising:
(a) coenzyme Q10 in the range of 50-1200 mg,
(b) vitamin C in the range of 50-2000 mg, and
(c) vitamin E in the range of 10-1000 mg.

2. The composition of claim 1, further comprising PPQ in the range of 10 to 300 mg.

3. The composition of claim 1, further comprising Resveratrol in the range of 20 to 300 mg.

4. A composition comprising:
(a) coenzyme Q10 in the range of 50-1200 mg,
(b) PPQ in the range of 10 to 300 mg, and
(c) Resveratrol in the range of 20 to 300 mg.

5. The composition of claim 1, wherein the composition further comprises vitamin D in the range of 200-4000 IU.

6. The composition of claim 4, wherein the composition further comprises vitamin D in the range of 200-4000 IU.

7. The composition of claim 1, wherein the composition further comprises folic acid in the range of 0.1 to 5 mg.

8. The composition of claim 4, wherein the composition further comprises folic acid in the range of 0.1 to 5 mg.

9. The composition of claim 1, wherein the composition further comprises vitamin B12 in the range of 2.0 mcg-1000.0 mcg.

10. The composition of claim 4 wherein the composition further comprises vitamin B12 in the range of 2.0 mcg-1000.0 mcg.

11. The composition of claim 1, wherein the composition comprises an oral daily dose in unit form.

12. The composition of claim 4, wherein the composition comprises an oral daily dose in unit form.

13. A composition for female fertility, the composition comprising coenzyme Q10; and vitamin C, vitamin E, PPQ, Resveratrol, or combinations thereof.

14. The composition of claim 13, wherein coenzyme Q10 is in the range of 50-1200 mg.

15. The composition of claim 13, wherein Vitamin C is in the range of 50-2000 mg.

16. The composition of claim 13, wherein Vitamin E is in the range of 10-1000 mg.

17. The composition of claim 13, wherein PPQ is in the range of 10 to 300 mg.

18. The composition of claim 13, wherein Resveratrol is in the range of 20 to 300 mg.

19. The composition of claim 13, comprising:
(a) coenzyme Q10 in the range of 50-1200 mg,
(b) vitamin C in the range of 50-2000 mg, and
(c) vitamin E in the range of 10-1000 mg.

20. The composition of claim 19, further comprising PPQ in the range of 10 to 300 mg.

21. The composition of claim 19, further comprising Resveratrol in the range of 20 to 300 mg.

22. The composition of claim 13, comprising:
(a) coenzyme Q10 in the range of 50-1200 mg,
(b) PPQ in the range of 10 to 300 mg, and
(c) Resveratrol in the range of 30 to 300 mg.

23. The composition of claim 19, wherein the composition further comprises Vitamin D in the range of 200-4000 IU.

24. The composition of claim 22, wherein the composition further comprises Vitamin D in the range of 200-4000 IU.

25. The composition of claim 19, wherein the composition further comprises folic acid in the range of 0.1 to 5 mg.

26. The composition of claim 22, wherein the composition further comprises folic acid in the range of 0.1 to 5 mg.

27. The composition of claim 19, wherein the composition further comprises vitamin B12 in the range of 2.0 mcg-1000.0 mcg.

28. The composition of claim 22, wherein the composition further comprises vitamin B12 in the range of 2.0 mcg-1000.0 mcg.

29. The composition of claim of claim 13, for prolonging fertility in women by reducing the number of free radicals.

30. The composition of claim 23, for enhancing fertility rates in women by increasing the expression of anti-Müllerian hormone.

31. The composition of claim 25, for promoting the development of the nervous system of a fetus.

32. The composition of claim 13, wherein the composition comprises an oral daily dose in unit form.

33. The composition of claim 11, wherein the oral daily unit dosage form is selected from the group consisting of: a capsule, a tablet, a chewable tablet, a liquid, a liquid-gel capsule, a gel capsule with dry powder, a vegetable capsule with dry powder, a dry powder, an effervescent tablet, and a gum-based chewable.

34. The composition of claim 12, wherein the oral daily unit dosage form is selected from the group consisting of: a capsule, a tablet, a chewable tablet, a liquid, a liquid-gel capsule, a gel capsule with dry powder, a vegetable capsule with dry powder, a dry powder, an effervescent tablet, and a gum-based chewable.

35. The composition of claim 32, wherein the oral daily unit dosage form is selected from the group consisting of: a capsule, a tablet, a chewable tablet, a liquid, a liquid-gel capsule, a gel capsule with dry powder, a vegetable capsule with dry powder, a dry powder, an effervescent tablet, and a gum-based chewable.

36. A pack comprising the oral daily dose in unit form as defined in any one of claims 11, 12 and 32, wherein the oral daily unit dosage form is selected from the group consisting of: individual capsules, tablets, chewable tablets, liquid-gel capsules, gel capsules, vegetable capsules, effervescent tablets and gum-based chewables.
